(19) United States
(12) Patent Application Publication
(10) Pub. No.: US 2024/0355420 A1
(43) Pub. Date: Oct. 24, 2024

(54) REANALYSIS BASED ON VERSION MANAGEMENT

(71) Applicant: Helix, Inc., San Mateo, CA (US)

(72) Inventors: Enakshi Singh, Burlingame, CA (US); Sharoni Jacobs, Mill Valley, CA (US); Emre Colak, San Mateo, CA (US)

(21) Appl. No.: 18/136,670

(22) Filed: Apr. 19, 2023

(51) Int. Cl. G16B 30/00; G16B 50/00

(52) U.S. Cl. CPC G16B 30/00; G16B 50/00

(57) ABSTRACT

A machine accesses first results of a first analysis performed by a first version of a first analytical tool on sequenced data of a genetic sample. A second analytical tool is to process results of the first analytical tool. The machine determines that a second analysis is to be performed by a second version of the first analytical tool upon the sequenced data of the genetic sample. A third analytical tool into which second results of the second analytical tool are to be inputted specifies a minimum version of the first analytical tool, and the determining that the second analysis is to be performed may be based on the first version failing to satisfy the minimum version, the second version satisfying the minimum version, or both. The machine then causes the second analysis to be performed by the second version of the first analytical tool on the sequenced data.

### TECHNICAL FIELD

The subject matter disclosed herein generally relates to the technical field of special-purpose machines that facilitate analysis of data by multiple analytical tools, including software-configured computerized variants of such special-purpose machines and improvements to such variants, and to the technologies by which such special-purpose machines become improved compared to other special-purpose machines that facilitate analysis of data by multiple analytical tools. Specifically, the present disclosure addresses systems and methods to facilitate reanalysis of data by an analytical tool.

### BACKGROUND

A machine may be configured to process data by causing (e.g., triggering, launching, invoking, or otherwise starting) one or more analytical tools (e.g., analytical software tools, such as analytical apps, analysis applications, or other data analyzing programs) to process some initially accessed data in a pipelined manner, where the results produced by one analytical tool become input for a subsequent analytical tool, and the results produced by the subsequent analytical tool, in turn, become input for a further subsequent analytical tool, and so on.

### BRIEF DESCRIPTION OF THE DRAWINGS

Some embodiments are illustrated by way of example and not limitation in the figures of the accompanying drawings.

FIG. 1 is a network diagram illustrating a network environment suitable for reanalysis based on version management, according to some example embodiments.

FIG. 2 is a block diagram illustrating components of a machine suitable for reanalysis based on version management, according to some example embodiments.

FIG. 3 is a block diagram illustrating components of a database suitable for reanalysis based on version management, according to some example embodiments.

FIGS. 4 and 5 are flowcharts illustrating operations of a machine in performing a method of reanalysis based on version management, according to some example embodiments.

FIG. 6 is a block diagram illustrating components of a machine, according to some example embodiments, able to read instructions from a machine-readable medium and perform any one or more of the methodologies discussed herein.

FIG. 7 is a block diagram illustrating a bioinformatics analysis system, according to some example embodiments.

FIG. 8 is a flowchart illustrating a method of using a bioinformatics analysis system, according to some example embodiments.

FIGS. 9-11 are flowcharts illustrating a method of intaking and preparing a genetic sample in a wet laboratory for bioinformatic analysis, according to some example embodiments.

### SUMMARY

An example machine configured in accordance with the present subject matter accesses first results of a first analysis performed by a first version of a first analytical tool on sequenced data (e.g., sequence data) of a genetic sample or on results of a preceding analytical tool. A second analytical tool may be available to process results of the first analytical tool. The machine may then determine that a second analysis is to be performed by a second version of the first analytical tool upon the same sequenced data of the genetic sample or the same results of the preceding analytical tool. A third analytical tool into which second results of the second analytical tool are to be inputted may specify a minimum version of the first analytical tool. The aforementioned determination that the second analysis is to be performed may be based on (e.g., in response to) the first version failing to satisfy the minimum version, the second version satisfying the minimum version, or a conjunction of both. The machine may then cause the second analysis to be performed by the second version of the first analytical tool on the same sequenced data of the genetic sample or on the same results of the preceding analytical tool.

### DETAILED DESCRIPTION

Example methods (e.g., algorithms) facilitate reanalysis of data based on version management, and example systems (e.g., special-purpose machines configured by special-purpose software) are configured to facilitate reanalysis of data based on version management. Examples merely typify possible variations. Unless explicitly stated otherwise, structures (e.g., structural components, such as modules) are optional and may be combined or subdivided, and operations (e.g., in a procedure, algorithm, or other function) may vary in sequence or be combined or subdivided. In the following description, for purposes of explanation, numerous specific details are set forth to provide a thorough understanding of various example embodiments. It will be evident to one skilled in the art, however, that the present subject matter may be practiced without these specific details.

As configured in accordance with the system and methods discussed herein, an analysis manager machine accesses first results of a first analysis that has been performed by a first version of a first analytical tool, using sequenced data of a genetic sample as input to the first analytical tool or using results of a preceding analytical tool as input to the first analytical tool. A second analytical tool is typically available to the analysis manager machine, configured to process one or more results of the first analytical tool, and configured to output second results.

The analysis manager machine then determines that a second analysis is to be performed by a second and different version of the first analytical tool, using the same sequenced data of the genetic sample as input or using the same results of the preceding analytical tool as input. A third analytical tool is typically available to the analysis manager machine, and the third analytical tool is configured to use the second results of the second analytical tool as input.

The third analytical tool may specify a minimum version of the first analytical tool, and accordingly, the above-described determination that the second analysis is to be performed may be based on (e.g., in response to) the first version of the first analytical tool failing to satisfy the minimum version, the second version of the first analytical tool satisfying the minimum version, or a conjunction (e.g., a co-occurrence) of both. The analysis manager machine may then cause the second analysis to be performed by the second version of the first analytical tool, using the same sequenced data of the genetic sample as input or using the same results of the preceding analytical tool as input. Further details of the analysis manager machine are discussed below.

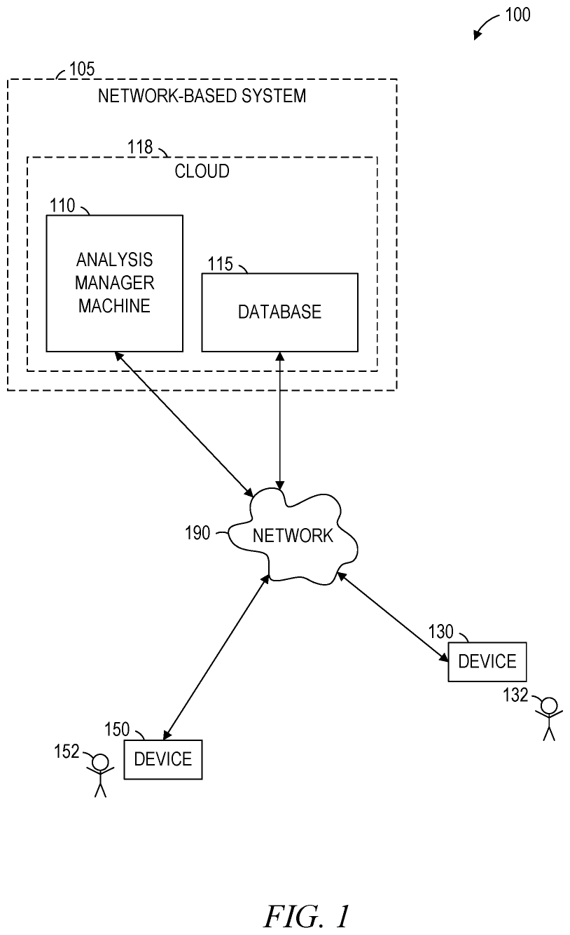

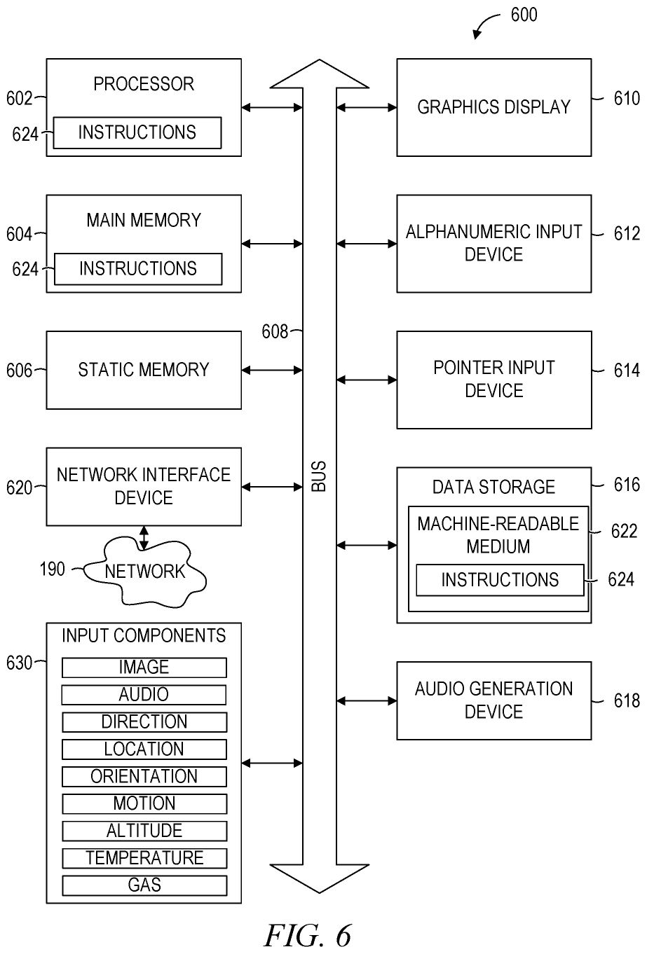

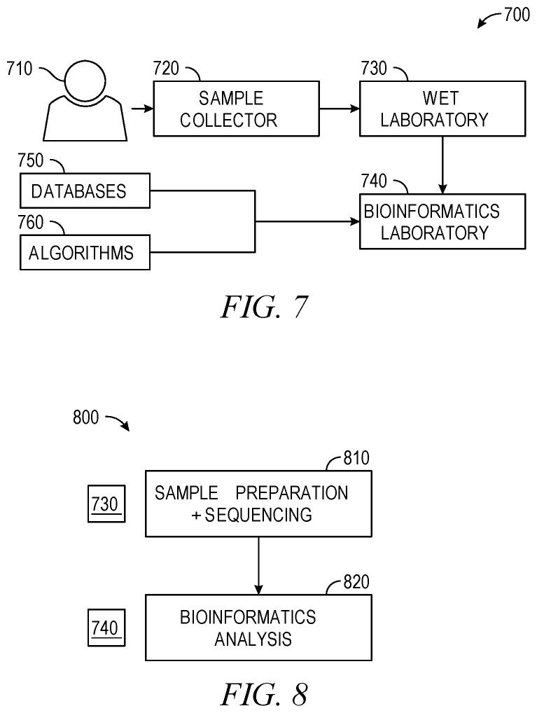

FIG. 1 is a network diagram illustrating a network environment 100 suitable for reanalysis based on version management, according to some example embodiments. The network environment 100 includes an analysis manager machine 110, a database 115, and devices 130 and 150, all communicatively coupled to each other via a network 190. The analysis manager machine 110, with or without the database 115, may form all or part of a cloud 118 (e.g., a geographically distributed set of multiple machines configured to function as a single server), which may form all or part of a network-based system 105 (e.g., a cloud-based server system configured to provide one or more network-based services to the devices 130 and 150). The analysis manager machine 110, the database 115, and the devices 130 and 150 may each be implemented in a special-purpose (e.g., specialized) computer system, in whole or in part, as described below with respect to FIG. 6. The network environment 100 in whole or any one or more portions thereof may be included in a bioinformatics laboratory, as described below with respect to FIGS. 7-11.

Also shown in FIG. 1 are users 132 and 152. One or both of the users 132 and 152 may be a human user (e.g., a human being), a machine user (e.g., a computer configured by a software program to interact with the device 130 or 150), or any suitable combination thereof (e.g., a human assisted by a machine or a machine supervised by a human). The user 132 is associated with the device 130 and may be a user of the device 130. For example, the device 130 may be a desktop computer, a vehicle computer, a home media system (e.g., a home theater system or other home entertainment system), a tablet computer, a navigational device, a portable media device, a smart phone, or a wearable device (e.g., a smart watch, smart glasses, smart clothing, or smart jewelry) belonging to the user 132. Likewise, the user 152 is associated with the device 150 and may be a user of the device 150. As an example, the device 150 may be a desktop computer, a vehicle computer, a home media system (e.g., a home theater system or other home entertainment system), a tablet computer, a navigational device, a portable media device, a smart phone, or a wearable device (e.g., a smart watch, smart glasses, smart clothing, or smart jewelry) belonging to the user 152.

Any of the systems or machines (e.g., databases and devices) shown in FIG. 1 may be, include, or otherwise be implemented in a special-purpose (e.g., specialized or otherwise non-conventional and non-generic) computer that has been modified to perform one or more of the functions described herein for that system or machine (e.g., configured or programmed by special-purpose software, such as one or more software modules of a special-purpose application, operating system, firmware, middleware, or other software program). For example, a special-purpose computer system able to implement any one or more of the methodologies described herein is discussed below with respect to FIG. 6, and such a special-purpose computer may accordingly be a means for performing any one or more of the methodologies discussed herein. Within the technical field of such special-purpose computers, a special-purpose computer that has been specially modified (e.g., configured by special-purpose software) by the structures discussed herein to perform the functions discussed herein is technically improved compared to other special-purpose computers that lack the structures discussed herein or are otherwise unable to perform the functions discussed herein. Accordingly, a special-purpose machine configured according to the systems and methods discussed herein provides an improvement to the technology of similar special-purpose machines.

As used herein, a “database” is a data storage resource and may store data structured in any of various ways, for example, as a text file, a table, a spreadsheet, a relational database (e.g., an object-relational database), a triple store, a hierarchical data store, a document database, a graph database, key-value pairs, or any suitable combination thereof. Moreover, any two or more of the systems or machines illustrated in FIG. 1 may be combined into a single system or machine, and the functions described herein for any single system or machine may be subdivided among multiple systems or machines.

The network 190 may be any network that enables communication between or among systems, machines, databases, and devices (e.g., between the analysis manager machine 110 and the device 130). Accordingly, the network 190 may be a wired network, a wireless network (e.g., a mobile or cellular network), or any suitable combination thereof. The network 190 may include one or more portions that constitute a private network, a public network (e.g., the Internet), or any suitable combination thereof. Accordingly, the network 190 may include one or more portions that incorporate a local area network (LAN), a wide area network (WAN), the Internet, a mobile telephone network (e.g., a cellular network), a wired telephone network (e.g., a plain old telephone service (POTS) network), a wireless data network (e.g., a WiFi network or WiMax network), or any suitable combination thereof. Any one or more portions of the network 190 may communicate information via a transmission medium. As used herein, “transmission medium” refers to any intangible (e.g., transitory) medium that is capable of communicating (e.g., transmitting) instructions for execution by a machine (e.g., by one or more processors of such a machine), and includes digital or analog communication signals or other intangible media to facilitate communication of such software.

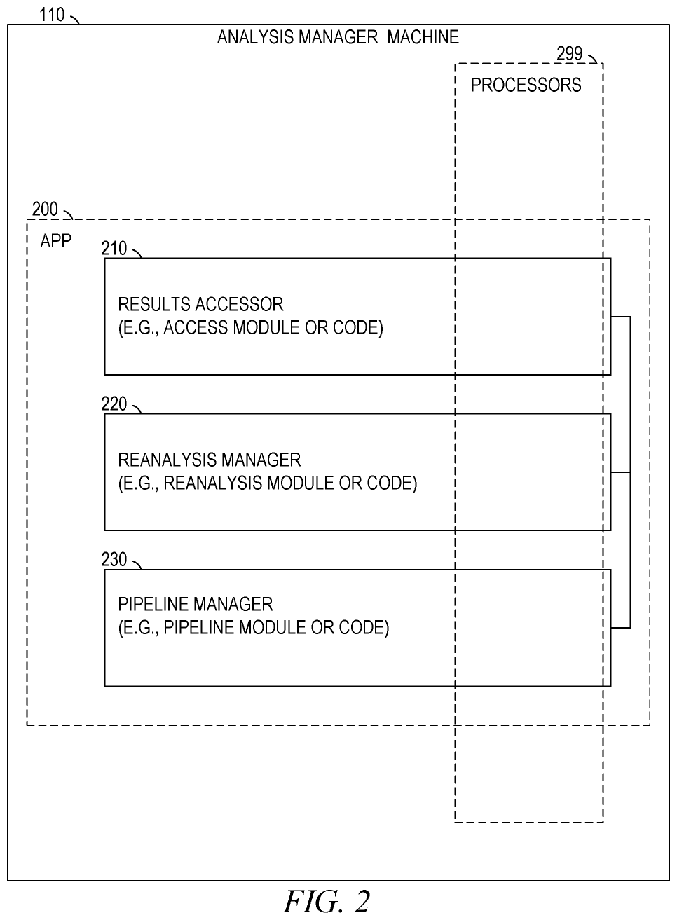

FIG. 2 is a block diagram illustrating components of the analysis manager machine 110, as configured for reanalysis based on version management, according to some example embodiments. The analysis manager machine 110 is shown as including a results accessor 210 (e.g., an access module or similarly suitable code), a reanalysis manager 220 (e.g., a reanalysis module or similarly suitable code), and a pipeline manager 230 (e.g., a pipeline module or similarly suitable code), all configured to communicate with each other (e.g., via a bus, shared memory, or a switch).

As shown in FIG. 2, the results accessor 210, the reanalysis manager 220, the pipeline manager 230, or any suitable combination thereof, may form all or part of an app 200 (e.g., a server app, a mobile app, or other app) that is stored (e.g., installed) on the analysis manager machine 110 (e.g., responsive to or otherwise as a result of data being via the network 190). Furthermore, one or more processors 299 (e.g., hardware processors, digital processors, or any suitable combination thereof) may be included (e.g., temporarily or permanently) in the app 200, the results accessor 210, the reanalysis manager 220, the pipeline manager 230, or any suitable combination thereof.

Any one or more of the components (e.g., modules) described herein may be implemented using hardware alone (e.g., one or more of the processors 299) or a combination of hardware and software. For example, any component described herein may physically include an arrangement of one or more of the processors 299 (e.g., a subset of or among the processors 299) configured to perform the operations described herein for that component. As another example, any component described herein may include software, hardware, or both, that configure an arrangement of one or more of the processors 299 to perform the operations described herein for that component. Accordingly, different components described herein may include and configure different arrangements of the processors 299 at different points in time or a single arrangement of the processors 299 at different points in time. Each component (e.g., module) described herein is an example of a means for performing the operations described herein for that component. Moreover, any two or more components described herein may be combined into a single component, and the functions described herein for a single component may be subdivided among multiple components. Furthermore, according to various example embodiments, components described herein as being implemented within a single system or machine (e.g., a single device) may be distributed across multiple systems or machines (e.g., multiple devices).

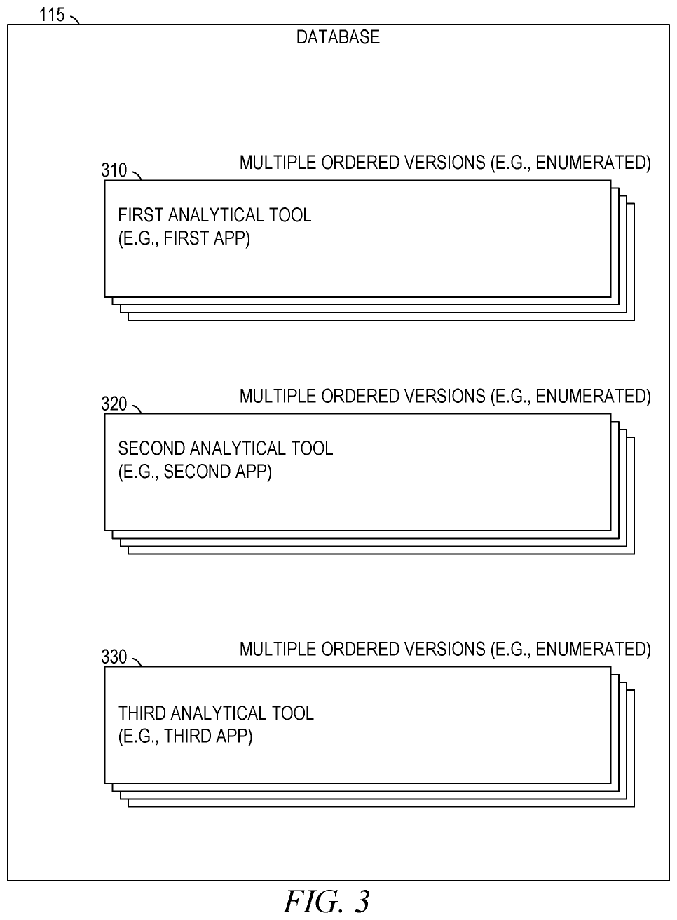

FIG. 3 is a block diagram illustrating components of the database 115, as configured for reanalysis based on version management, according to some example embodiments. The database 115 stores and makes available (e.g., for access, initiation, execution, or other usage by the analysis manager machine 110) various analytical tools, including various versions of each analytical tool. As shown in FIG. 3, the database 115 stores and makes available a first analytical tool 310 (e.g., a first analytical app), including multiple ordered (e.g., enumerated, sequential, or otherwise ordered) versions of the first analytical tool. For example, the database 115 may store and make available a first version (e.g., version 1.0) of the first analytical tool, as well as a second version (e.g., version 2.5) of the same first analytical tool. In various example embodiments, the database 115 stores a most recent version (e.g., a latest version or a current version) of the first analytical tool 310 and stores information (e.g., indicators, which may be in metadata, or other records) that indicates one or more previous versions of the first analytical tool 310.

The database 115 may also store and make available a second analytical tool 320 (e.g., a second analytical app), including multiple ordered (e.g., enumerated, sequential, or otherwise ordered) versions of the same second analytical tool. In some example embodiments, the database 115 stores a most recent version (e.g., a latest version or a current version) of the second analytical tool 320 and stores information (e.g., indicators, which may be in metadata, or other records) that indicates one or more previous versions of the second analytical tool 320. The database 115 may also store and make available a third analytical tool 330 (e.g., a third analytical app), including multiple ordered (e.g., enumerated, sequential, or otherwise ordered) versions of the same third analytical tool. In certain example embodiments, the database 115 stores a most recent version (e.g., a latest version or a current version) of the third analytical tool 330 and stores information (e.g., indicators, which may be in metadata, or other records) that indicates one or more previous versions of the third analytical tool 330. One or more versions of the third analytical tool 330 may specify (e.g., indicate within its corresponding metadata, which may also be stored by the database 115 and made available by the database 115) a minimum version of the first analytical tool (e.g., a lowest ordered version of the first analytical tool whose output will be compatible with or otherwise usable by that version of the third analytical tool).

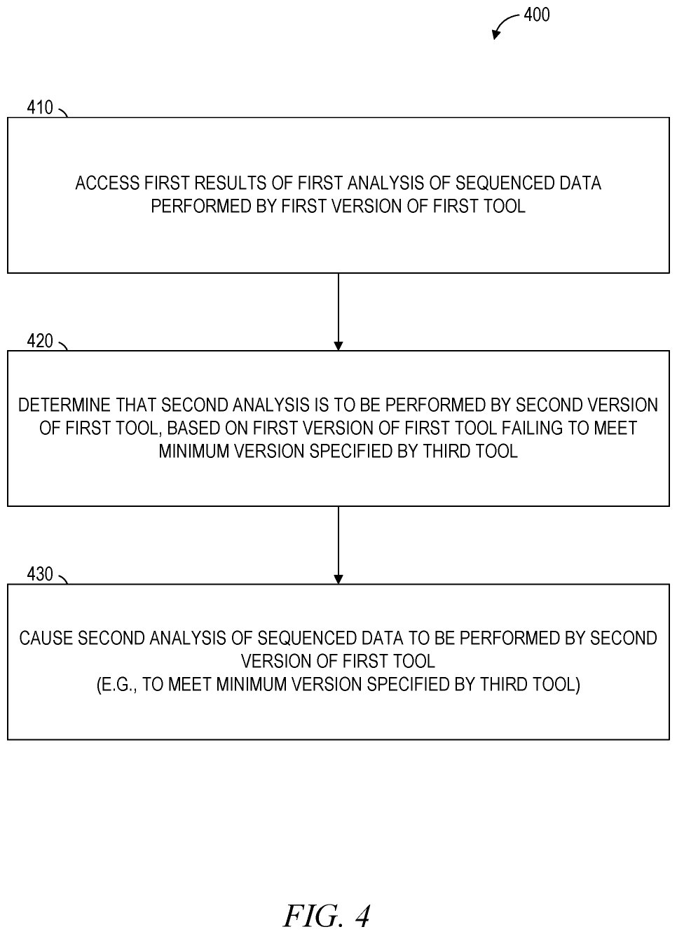

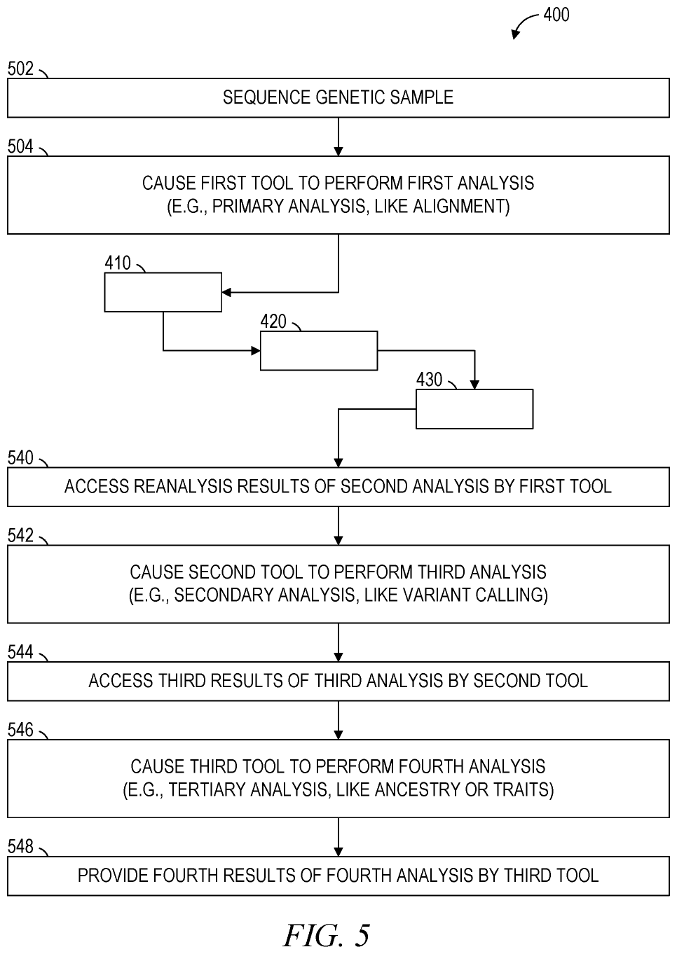

FIGS. 4 and 5 are flowcharts illustrating operations of the analysis manager machine 110 in performing a method 400 of reanalysis based on version management, according to some example embodiments. Operations in the method 400 may be performed by the analysis manager machine 110, using components (e.g., modules) described above with respect to FIG. 2, using one or more processors (e.g., microprocessors or other hardware processors), or using any suitable combination thereof. As shown in FIG. 4, the method 400 includes operations 410, 420, and 430.

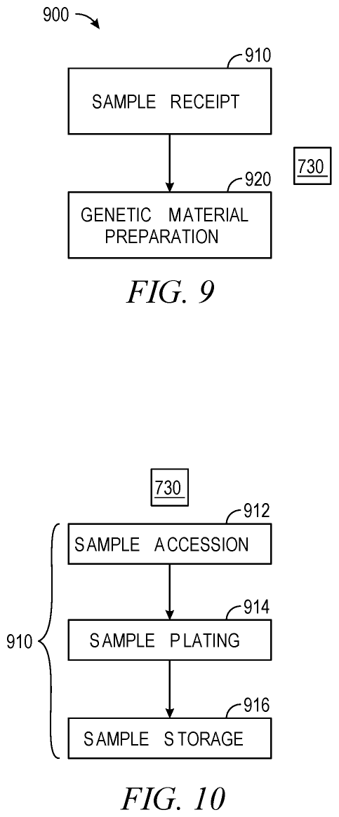

In operation 410, the results accessor 210 accesses (e.g., receives, reads, retrieves, or otherwise obtains) first results of a first analysis performed by a first version of a first analytical tool (e.g., first analytical tool 310) on previously sequenced data of a genetic sample. For example, the first analytical tool may perform alignment on the sequenced data. In alternative example embodiments, the first analysis is performed on results of a preceding analytical tool and not performed directly on sequenced data of the genetic sample (e.g., as output from a genetic sequencing operation). The first results may indicate (e.g., within its corresponding metadata or within other associated data) which version (e.g., the first version) of the first analytical tool (e.g., first analytical tool 310) produced the first results. As noted above, a second analytical tool (e.g., second analytical tool 320) is available to process results of the first analytical tool. In some example embodiments, the first analytical tool is a primary analysis tool that is configured to perform alignment (e.g., as detailed below with respect to FIGS. 9-11) of the sequenced data of the genetic sample. The resulting aligned sequenced data may be stored or communicated in a Binary Alignment Map (BAM) file. Metadata that identifies the version of the first analytical tool may be stored in the BAM file, stored with the BAM file, mapped (e.g., linked) to the BAM file, or otherwise associated with the BAM file (e.g., by the reanalysis manager machine 110, the app 200, any portion thereof, or suitable combination thereof).

In operation 420, the reanalysis manager 220 determines (e.g., decides, concludes, or otherwise obtains a decision) that a second analysis (e.g., as a reanalysis of the same input data as in the first analysis) is to be performed by a second version of the first analytical tool (e.g., first analytical tool 310) upon the sequenced data of the genetic sample. In alternative example embodiments, the second analysis is to be performed on the same results of the preceding analytical tool and not performed directly on sequenced data of the genetic sample (e.g., as output from a genetic sequencing operation). As noted above, a third analytical tool (e.g., third analytical tool 330) into which second results of the second analytical tool (e.g., second analytical tool 320) are to be inputted specifies a minimum version of the first analytical tool (e.g., for compatibility with one or more versions of the third analytical tool). In operation 420, the determining that the second analysis is to be performed may be based on (e.g., responsive to) the first version failing to satisfy the minimum version, the second version satisfying the minimum version, or a conjunction (e.g., a co-occurrence) of both.

In operation 430, the pipeline manager 230 causes (e.g., by triggering, invoking, launching, initiating, executing, or otherwise starting) the second analysis to be performed by the second version of the first analytical tool on the sequenced data of the genetic sample. In alternative example embodiments, the second analysis is caused to be performed on the same results of the preceding analytical tool and not performed directly on sequenced data of the genetic sample (e.g., as output from a genetic sequencing operation). According to some example embodiments, the second analysis includes an improvement that was absent from the first analysis, and in various example embodiments, the improvement includes analyzing an additional portion of a gene, analyzing an additional gene, omitting analysis of a portion of a gene, omitting analysis of a gene, analyzing more total portions of genes, analyzing more total genes, analyzing fewer total portions of genes, analyzing fewer total genes, or any suitable combination thereof.

As shown in FIG. 5, in addition to any one or more of the operations previously described, the method 400 may include one or more of operations 502, 504, 540, 542, 544, 546, and 548. One or more of operations 502 and 504 may be performed prior to operation 410. One or more of operations 540, 542, 544, 546, and 548 may be performed after operation 430.

In operation 502, a sequencing tool (e.g., a machine configured to function as described below with respect to FIGS. 9-11) sequences a genetic sample. The sequencing tool may receive a genetic sample as input, sequence the genetic sample, and provide sequenced data of the genetic sample as output.

In operation 504, the pipeline manager 230 causes the first analytical tool (e.g., first analytical tool 310) to perform the first analysis on the sequenced data of the genetic sample. In alternative example embodiments, the first analysis is performed by the first analytical tool on results of a preceding analytical tool and not performed directly on sequenced data of the genetic sample (e.g., as output from a genetic sequencing operation).

In operation 540, the results accessor 210 assesses the second results of the second analysis (e.g., the reanalysis) that was caused, in operation 430, to be performed by the second version of the first analytical tool (e.g., first analytical tool 310). As noted above, the second analysis may be performed on the same sequenced data of the genetic sample as used to perform the first analysis. In alternative example embodiments, the second analysis is performed on the same results of the preceding analytical tool as used to perform the first analysis.

In operation 542, with the second results (e.g., obtained from the reanalysis initiated in operation 430) now having been accessed, the pipeline manager 230 causes a third analysis to be performed by the second analytical tool (e.g., second analytical tool 320) on the now accessed second results (e.g., from the reanalysis). In certain example embodiments, the second analytical tool is a secondary analysis tool that is configured to perform variant calling (e.g., as detailed below with respect to FIGS. 9-11) on the first results of the first analytical tool (e.g., first analytical tool 310). The resulting variant-called aligned sequenced data may be stored or communicated in a Variant Call Formant (VCF) file. Metadata that identifies the version of the second analytical tool may be stored in the VCF file, stored with the VCF file, mapped (e.g., linked) to the VCF file, or otherwise associated with the VCF file (e.g., by the reanalysis manager machine 110, the app 200, any portion thereof, or suitable combination thereof).

According to some example embodiments, the second results include an improvement that was absent from the first results, and in various example embodiments, the improvement includes a correction to an error, an additional datum, a datum with increased precision, a datum with increased accuracy, a data format supported by the third analytical tool, a file format supported by the third analytical tool, a metadata type supported by the third analytical tool, or a metadata value supported by the third analytical tool, or any suitable combination thereof.

In operation 544, the results accessor 210 assesses third results of the third analysis that was caused, in operation 542, to be performed by the second analytical tool (e.g., second analytical tool 320). As noted above, the third analysis may be performed on the results of the second analysis (e.g., the reanalysis) that was caused, in operation 430.

In operation 546, with the third results now having been accessed, the pipeline manager 230 causes a fourth analysis to be performed by the third analytical tool (e.g., third analytical tool 330) on the now accessed third results. According to various example embodiments, the third analytical tool is a tertiary analysis tool configured to process secondary results outputted by the second analytical tool (e.g., second analytical tool 320) based on processing the first results of the first analytical tool (e.g., first analytical tool 310). For example, the tertiary analysis tool may be configured to process the secondary results by performing ancestry analysis, traits analysis, medical diagnosis, or any suitable combination thereof.

In operation 548, the results accessor 210 accesses and provides fourth results of the fourth analysis that was caused, in operation 546, to be performed by the third analytical tool (e.g., third analytical tool 330). As noted above, the fourth analysis may be performed on the results of the third analysis that was caused, in operation 542, to be performed by the second analytical tool (e.g., second analytical tool 320). The fourth results may be provided to the database 115, one or more of the devices 130 and 150, another machine communicatively coupled to the analysis manager machine 110 via the network 190, or any suitable combination thereof.

According to various example embodiments, one or more of the methodologies described herein may facilitate reanalysis of data based on version management. Moreover, one or more of the methodologies described herein may facilitate ensuring that a tertiary analysis tool is provided with suitable input based on one or more improvements included in upstream results outputted from a primary analysis tool. Hence, one or more of the methodologies described herein may facilitate compatibility of multiple analytical tools in a pipeline of analyses, as well as management of such compatibility and automated invocation of reanalyses to conserve time, power, and other resources, compared to capabilities of pre-existing systems and methods.

For example, BAM files, VCF files, or both, may be obtained (e.g., acquired or accumulated) over time for a population of patients, as those patients undergo screening or other genetic tests, and a subset of this population may be appropriate for additional screening or other genetic tests. For a patient in this subset, a data analyst or a medical practitioner may indicate (e.g., to app 200 or a portion thereof) a type of additional screening or other genetic test to be performed on that patient. A system (e.g., including the reanalysis manager machine 110, the app 200, any portion thereof, or any suitable combination thereof) may be configured to access information (e.g., stored in the database 115 or any other part of the network-based system 105) that indicates a minimum version for a genetic testing tool available to perform the additional screening or other genetic test. The minimum version may vary depending on the specific additional genetic test to be performed. For example, population screening tests may have different minimum versions for alignment tools, for variant calling tools, for cardiac testing tools, and for pharmacogenomic testing tools. Such a system may be configured to selectively cause a reanalysis (e.g., as discussed above) to be performed, based on one or more of the minimum versions associated with the additional genetic test to be performed. In this manner, patients in need of additional genetic testing may receive results of a desired quality as quickly as possible. Specifically, results obtained from analytical tools that meet minimum versions may be provided quickly, while results obtained from analytical tools the do not meet minimum versions may be regenerated using analytical tools that do meet minimum versions. This may beneficially reduce wait time for such patients, while also enhancing the quality of results provide to such patients.

When these effects are considered in aggregate, one or more of the methodologies described herein may obviate a need for certain efforts or resources that otherwise would be involved in reanalysis of data based on version management. Efforts expended by a user in determining that reanalysis of data is to be performed may be reduced by use of (e.g., reliance upon) a special-purpose machine that implements one or more of the methodologies described herein. Computing resources used by one or more systems or machines (e.g., within the network environment 100) may similarly be reduced (e.g., compared to systems or machines that lack the structures discussed herein or are otherwise unable to perform the functions discussed herein). Examples of such computing resources include processor cycles, network traffic, computational capacity, main memory usage, graphics rendering capacity, graphics memory usage, data storage capacity, power consumption, and cooling capacity.

FIG. 6 is a block diagram illustrating components of a machine 600, according to some example embodiments, able to read instructions 624 from a machine-readable medium 622 (e.g., a non-transitory machine-readable medium, a machine-readable storage medium, a computer-readable storage medium, or any suitable combination thereof) and perform any one or more of the methodologies discussed herein, in whole or in part. Specifically, FIG. 6 shows the machine 600 in the example form of a computer system (e.g., a computer) within which the instructions 624 (e.g., software, a program, an application, an applet, an app, or other executable code) for causing the machine 600 to perform any one or more of the methodologies discussed herein may be executed, in whole or in part.

In alternative embodiments, the machine 600 operates as a standalone device or may be communicatively coupled (e.g., networked) to other machines. In a networked deployment, the machine 600 may operate in the capacity of a server machine or a client machine in a server-client network environment, or as a peer machine in a distributed (e.g., peer-to-peer) network environment. The machine 600 may be a server computer, a client computer, a personal computer (PC), a tablet computer, a laptop computer, a netbook, a cellular telephone, a smart phone, a set-top box (STB), a personal digital assistant (PDA), a web appliance, a network router, a network switch, a network bridge, or any machine capable of executing the instructions 624, sequentially or otherwise, that specify actions to be taken by that machine. Further, while only a single machine is illustrated, the term “machine” shall also be taken to include any collection of machines that individually or jointly execute the instructions 624 to perform all or part of any one or more of the methodologies discussed herein.

The machine 600 includes a processor 602 (e.g., one or more central processing units (CPUs), one or more graphics processing units (GPUs), one or more digital signal processors (DSPs), one or more application specific integrated circuits (ASICs), one or more radio-frequency integrated circuits (RFICs), or any suitable combination thereof), a main memory 604, and a static memory 606, which are configured to communicate with each other via a bus 608. The processor 602 contains solid-state digital microcircuits (e.g., electronic, optical, or both) that are configurable, temporarily or permanently, by some or all of the instructions 624 such that the processor 602 is configurable to perform any one or more of the methodologies described herein, in whole or in part. For example, a set of one or more microcircuits of the processor 602 may be configurable to execute one or more modules (e.g., software modules) described herein. In some example embodiments, the processor 602 is a multicore CPU (e.g., a dual-core CPU, a quad-core CPU, an 8-core CPU, or a 128-core CPU) within which each of multiple cores behaves as a separate processor that is able to perform any one or more of the methodologies discussed herein, in whole or in part. Although the beneficial effects described herein may be provided by the machine 600 with at least the processor 602, these same beneficial effects may be provided by a different kind of machine that contains no processors (e.g., a purely mechanical system, a purely hydraulic system, or a hybrid mechanical-hydraulic system), if such a processor-less machine is configured to perform one or more of the methodologies described herein.

The machine 600 may further include a graphics display 610 (e.g., a plasma display panel (PDP), a light emitting diode (LED) display, a liquid crystal display (LCD), a projector, a cathode ray tube (CRT), or any other display capable of displaying graphics or video). The machine 600 may also include an alphanumeric input device 612 (e.g., a keyboard or keypad), a pointer input device 614 (e.g., a mouse, a touchpad, a touchscreen, a trackball, a joystick, a stylus, a motion sensor, an eye tracking device, a data glove, or other pointing instrument), a data storage 616, an audio generation device 618 (e.g., a sound card, an amplifier, a speaker, a headphone jack, or any suitable combination thereof), and a network interface device 620.

The data storage 616 (e.g., a data storage device) includes the machine-readable medium 622 (e.g., a tangible and non-transitory machine-readable storage medium) on which are stored the instructions 624 embodying any one or more of the methodologies or functions described herein. The instructions 624 may also reside, completely or at least partially, within the main memory 604, within the static memory 606, within the processor 602 (e.g., within the processor's cache memory), or any suitable combination thereof, before or during execution thereof by the machine 600. Accordingly, the main memory 604, the static memory 606, and the processor 602 may be considered machine-readable media (e.g., tangible and non-transitory machine-readable media). The instructions 624 may be transmitted or received over the network 190 via the network interface device 620. For example, the network interface device 620 may communicate the instructions 624 using any one or more transfer protocols (e.g., hypertext transfer protocol (HTTP)).

In some example embodiments, the machine 600 may be a portable computing device (e.g., a smart phone, a tablet computer, or a wearable device) and may have one or more additional input components 630 (e.g., sensors or gauges). Examples of such input components 630 include an image input component (e.g., one or more cameras), an audio input component (e.g., one or more microphones), a direction input component (e.g., a compass), a location input component (e.g., a global positioning system (GPS) receiver), an orientation component (e.g., a gyroscope), a motion detection component (e.g., one or more accelerometers), an altitude detection component (e.g., an altimeter), a temperature input component (e.g., a thermometer), and a gas detection component (e.g., a gas sensor). Input data gathered by any one or more of these input components 630 may be accessible and available for use by any of the modules described herein (e.g., with suitable privacy notifications and protections, such as opt-in consent or opt-out consent, implemented in accordance with user preference, applicable regulations, or any suitable combination thereof).

As used herein, the term “memory” refers to a machine-readable medium able to store data temporarily or permanently and may be taken to include, but not be limited to, random-access memory (RAM), read-only memory (ROM), buffer memory, flash memory, and cache memory. While the machine-readable medium 622 is shown in an example embodiment to be a single medium, the term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of carrying (e.g., storing or communicating) the instructions 624 for execution by the machine 600, such that the instructions 624, when executed by one or more processors of the machine 600 (e.g., processor 602), cause the machine 600 to perform any one or more of the methodologies described herein, in whole or in part. Accordingly, a “machine-readable medium” refers to a single storage apparatus or device, as well as cloud-based storage systems or storage networks that include multiple storage apparatus or devices. The term “machine-readable medium” shall accordingly be taken to include, but not be limited to, one or more tangible and non-transitory data repositories (e.g., data volumes) in the example form of a solid-state memory chip, an optical disc, a magnetic disc, or any suitable combination thereof.

A “non-transitory” machine-readable medium, as used herein, specifically excludes propagating signals per se. According to various example embodiments, the instructions 624 for execution by the machine 600 can be communicated via a carrier medium (e.g., a machine-readable carrier medium). Examples of such a carrier medium include a non-transient carrier medium (e.g., a non-transitory machine-readable storage medium, such as a solid-state memory that is physically movable from one place to another place) and a transient carrier medium (e.g., a carrier wave or other propagating signal that communicates the instructions 624).

Certain example embodiments are described herein as including modules. Modules may constitute software modules (e.g., code stored or otherwise embodied in a machine-readable medium or in a transmission medium), hardware modules, or any suitable combination thereof. A “hardware module” is a tangible (e.g., non-transitory) physical component (e.g., a set of one or more processors) capable of performing certain operations and may be configured or arranged in a certain physical manner. In various example embodiments, one or more computer systems or one or more hardware modules thereof may be configured by software (e.g., an application or portion thereof) as a hardware module that operates to perform operations described herein for that module.

In some example embodiments, a hardware module may be implemented mechanically, electronically, hydraulically, or any suitable combination thereof. For example, a hardware module may include dedicated circuitry or logic that is permanently configured to perform certain operations. A hardware module may be or include a special-purpose processor, such as a field programmable gate array (FPGA) or an ASIC. A hardware module may also include programmable logic or circuitry that is temporarily configured by software to perform certain operations. As an example, a hardware module may include software encompassed within a CPU or other programmable processor. It will be appreciated that the decision to implement a hardware module mechanically, hydraulically, in dedicated and permanently configured circuitry, or in temporarily configured circuitry (e.g., configured by software) may be driven by cost and time considerations.

Accordingly, the phrase “hardware module” should be understood to encompass a tangible entity that may be physically constructed, permanently configured (e.g., hardwired), or temporarily configured (e.g., programmed) to operate in a certain manner or to perform certain operations described herein. Furthermore, as used herein, the phrase “hardware-implemented module” refers to a hardware module. Considering example embodiments in which hardware modules are temporarily configured (e.g., programmed), each of the hardware modules need not be configured or instantiated at any one instance in time. For example, where a hardware module includes a CPU configured by software to become a special-purpose processor, the CPU may be configured as respectively different special-purpose processors (e.g., each included in a different hardware module) at different times. Software (e.g., a software module) may accordingly configure one or more processors, for example, to become or otherwise constitute a particular hardware module at one instance of time and to become or otherwise constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory (e.g., a memory device) to which it is communicatively coupled. A further hardware module may then, at a later time, access the memory to retrieve and process the stored output. Hardware modules may also initiate communications with input or output devices, and can operate on a resource (e.g., a collection of information from a computing resource).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module in which the hardware includes one or more processors. Accordingly, the operations described herein may be at least partially processor-implemented, hardware-implemented, or both, since a processor is an example of hardware, and at least some operations within any one or more of the methods discussed herein may be performed by one or more processor-implemented modules, hardware-implemented modules, or any suitable combination thereof.

Moreover, such one or more processors may perform operations in a “cloud computing” environment or as a service (e.g., within a “software as a service” (SaaS) implementation). For example, at least some operations within any one or more of the methods discussed herein may be performed by a group of computers (e.g., as examples of machines that include processors), with these operations being accessible via a network (e.g., the Internet) and via one or more appropriate interfaces (e.g., an application program interface (API)). The performance of certain operations may be distributed among the one or more processors, whether residing only within a single machine or deployed across a number of machines. In some example embodiments, the one or more processors or hardware modules (e.g., processor-implemented modules) may be located in a single geographic location (e.g., within a home environment, an office environment, or a server farm). In other example embodiments, the one or more processors or hardware modules may be distributed across a number of geographic locations.

FIG. 7 is a diagram of an example bioinformatics analysis system 700, while FIG. 8 is a flowchart illustrating an example method 800 of using such a bioinformatics analysis system 700.

The system 700 can include both physical or “wet” laboratory components and bioinformatics components. For example, the system 700 can interact with patients 710, from whom biological samples can be collected, in addition to sample collectors 720, which may be, for example, doctors, pharmacies, or other appropriate places where patient samples can be taken. The system 700 includes a wet laboratory 730 which is positioned to receive the biological samples and process those samples, such as at operation 810 of the method 800, to produce sequenced genetic material for analysis, such as at operation 820 of the method 800. Method operations for sample receipt, handling (e.g., accession), and sequencing, are discussed in detail below with reference to FIGS. 9, 10, and 11. The system 700 also includes a bioinformatics laboratory 740, which may be communicatively coupled (e.g., via one or more networks) to one or more databases 750 and configured to apply or otherwise use one or more algorithms 760.

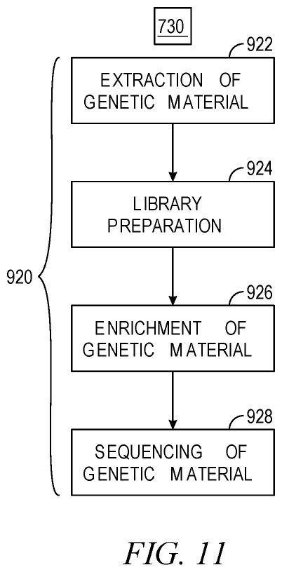

FIG. 9 is a flowchart illustrating a method 900 of intaking and preparing a genetic sample in a wet laboratory for bioinformatic analysis. The method 900 can include two primary portions: receiving the samples (operation 910) and preparing genetic material (operation 920). FIG. 10 illustrates portions of operation 910 of the method 900, including operations 912, 914, and 916 that result in intake of a genetic sample in a wet laboratory for bioinformatic analysis. FIG. 11 illustrates portions of operation 920 of the method 900, including operations 922, 924, 926, and 928 that result in preparation of a genetic sample in a wet laboratory for bioinformatic analysis.

The method 900 can begin with sample collection. For example, the samples can be collected by receiving a nasal swab, blood, saliva, or other material potentially containing genetic material indicative of a pathogen. The pathogen under study can be, for example, a ribonucleic acid (RNA) virus, such as SARS-CoV-2 or HIV, an adenovirus, or another type of pathogen with multiple variants having genetic material that could recombine for recombination and coinfection analysis.

Accessioning Samples. Once received at the laboratory, at operation 912, the samples can be accessioned, that is, prepared for later laboratory processes. For example, accessioning can include receiving a batch of samples. A batch of samples can include, for example, hundreds of individual samples or thousands of individual samples. Each sample can be retained in a sample container. For example, test tubes can be used to store each of the samples. The sample containers can be sealed to help prevent environmental exposure and prevent sample co-mingling. For example, the sample containers may be sealed via a cap that is threaded, glued, press-fit, or otherwise affixed via appropriate sealing mechanism. When the samples are received in a batch, the corresponding sample containers may also include one or more remnants of a sampling tool, such as a swab used to collect the sample.

In some cases, the sample containers may be accompanied by Customer Sample Identifiers (CSI) such as by a component affixed to or integrated with the sample container. Such a CSI can uniquely distinguish individual sample containers from other sample containers being received. For example, a CSI may uniquely distinguish a sample from other samples in the same batch, other samples received on the same date, or other samples received from the same customer. Such CSI can be provided as a label such as a bar code or a quick response (QR) code, a chip such as a radio frequency identifier (RFID), or another type of visual, transmission-generating, or other component affixed to or integrated with the sample container.

In some cases, the sample containers can be further sealed in an external container, such as a bag. External containers can help prevent contamination of samples, such as by preventing biological material from the samples contacting other or external surfaces. An external container can also help prevent cross-contamination between samples. Moreover, when a sample includes blood or a pathogen, the external container can provide an additional barrier to protect technicians who may handle the samples. The external container can additionally include documentation correlating to the CSI, such as information on the patient from which the sample was sourced, information indicating circumstances of sampling, for example, a sampling date, a sampling method, a location that the sample was acquired, a name or title for a person who performed the sampling, other information, or any suitable combination thereof.

In some cases, the samples can be in a chemical solution. For example, the sample may be prepared in an aqueous solution, such as a saline solution. In some cases, the samples can include a bodily fluid such as saliva, mucus, blood, or other. In an example, the sample can have a volume of about 2 mL, of about 3 mL, of about 4 mL, or of about 5 mL.

The samples include genetic material. For example, the samples can include deoxyribonucleic acid (DNA) or RNA. In an example, the genetic material is one or more of many constituent components within the sample. For example, one portion of the genetic material may exist within the nuclei or mitochondria of white blood cells that are included within the sample. In another example, another portion of the genetic material may exist within viruses or bacteria within the sample. In these types of examples, the genetic material is not yet isolated from the remaining constituent components of the sample. Thus, the genetic material should be isolated.

To begin isolating the genetic material, batches of the samples can be heated in ovens to facilitate cell lysis. The temperature and duration of heating can be chosen such that pathogenic material within the samples is rendered harmless, such that cellular lysis occurs, or both. For example, the samples can be heated at a temperature of between about 40° C. and 80° C., or at a temperature of between about 15° C. and 200° C., or at another appropriate temperature range. The samples can be heated for a time period of about 30 minutes, or for a time period of about 50 minutes, or for another appropriate time period. In some cases, such as where the samples are the contents of a blood draw, the heating operation may be skipped.

After heating, the batches of samples can be removed from the ovens. In an example, sample containers can be removed from external containers, such as by cutting open the external containers. The sample containers can be inspected, either in a manual, automated, or semi-automated fashion. For example, a technician or an automated system can determine the CSI for the sample and compare the CSI to documentation accompanying the batch. If there is a discrepancy between the CSIs on the sample container and in the documentation, the sample may be flagged as having an error condition. Similarly, if the CSI on the sample container is damaged (such as by abrasion, heat-damage, or water-damage) and has become unreadable, the sample may be flagged as having an error condition.

In some cases, the technician or automated system can further inspect the contents of the sample container, such as visually. If the sample does not include expected constituent components, then the sample can be flagged as having an error condition. For example, if the sample includes a fluid that is not permitted (such as extraneous blood), includes an entire swab or no swab, is within a fractured or broken sample container, is outside of an expected range of volume (e.g., between two and five milliliters), or other condition, then the sample can be flagged as having an error condition.

Subsequently, samples that have not been flagged with an error condition can proceed to sample integration. Here, the sample can be assigned a Laboratory Sample Identifier (LSI). Such an LSI can uniquely identify the sample from other samples received in the same batch, received on the same day, processed in the same laboratory, handled by the same company for sequencing, or combinations thereof. The LSI can be stored in a laboratory sample database and uniquely correlated to the CSI for the sample. The LSI can be associated with any error codes reported from the sample. Both the CSI and the LSI can both be applied to the sample container.

Sample Plating. Once accessioned, the samples can be plated at operation 914. At this point, the samples have been successfully integrated into the laboratory environment and are ready for analytics. At this point, the samples can be prepared for transfer to a sample microplate. The sample microplate can be labeled with a unique identifier, which can distinguish the sample microplate from other sample microplates. For example, the sample microplate can be a solid body with about 50 wells to about 400 wells, distributed across rows and columns, each well having a capacity of about 30 μL to about 300 μL. In other examples, different size microplates with a different number of wells at varying volumes can be used.

The samples to be used on the microplate may be racked and the rack may be assigned an identifier, such as to allow a technician to understand which samples correspond to which LSIs. The technician may unseal the sample, such as by a manual, automated, or semi-automated tool to efficiently open the sample container. The tooling may, for example, unscrew, cut, or drill each sample container, to make the sample within available for physical transfer to the sample microplate.

The samples can then be transferred to the microplate, such as by an automated robot that operates an end effector in accordance with one or more programs for effective transfer of the samples. This can be done, for example, with a combination of actuators, piezoelectric elements, pressure systems, or other components operating the end effector of the robot. The end effector can uptake portions of the samples in micropipettes and transfer those samples to the corresponding wells in the microplate. In some cases, disposable tips can be used. In some cases, portions of the samples can be transferred. In some cases, reagents can be added to the samples. In some cases, controls can be included in the microplate. The sample microplate, once completed, can be transferred for further processing in the laboratory.

Sample Storage. After plating, the samples can be stored at operation 916. In some cases, accessioned samples, plated samples, or other samples are stored for later use. In this case, they can be stored at room temperature or can be cryogenically frozen and arranged on racks for later retrieval. Samples can be preserved for periods of days or years to allow later rapid retesting.

Extraction of Genetic Material. When genetic analysis is desired, the genetic material of the samples can be extracted for sequencing at operation 922. In some examples, a reagent can be applied to sample wells to lyse cells therein to expose genetic material.

Additionally, aspirating and dispensing reagents can be used to selectively bind genetic material released from lysed cells. In some examples, this can include applying a bead to the well. In this case, the beads can, for example, be magnetic beads that selectively bind to the genetic material. This can help allow for isolation and purification of the genetic material at the bead, leaving contaminants in the solution. In an example, a magnetic bead can be magnetically drawn to a magnetic base at or under the sample microplate. In this case, after the genetic material has been drawn to the bead, a flushing operation can be performed to wash away remaining fluid, helping to remove impurities.

In some examples, fluid can be added or removed from wells, such as to concentrate or elute the genetic material. Fluid can be transferred from the wells of the sample microplate to a genome stock microplate. In an example, a portion of fluid can be removed from each well for quality control purposes. This can, for example, be used to determine concentration of genetic material therein.

Library Preparation. After extraction of the genetic material, a library can be prepared using the contents of the genome stock microplate at operation 924. For example, the bead for each well, including ionically bonded genetic material, can be transferred to a distinct well of a library preparation microplate. The library preparation microplate can include an identifier. The LSI associated with each well on the sample microplate can be mapped to a corresponding well on the library preparation microplate. The library preparation microplate may be transferred to a new portion of the laboratory to help prevent amplified genetic material from entering portions of the laboratory where genetic material has not been amplified, which could result in contamination.

A reagent can be applied to each well of the library preparation microplate. The reagent can ionically bond to the surface of the bead within the well more strongly than the genetic material. This helps release the genetic material from the surface of the bead of each well, enabling the genetic material to be chemically interacted with.

Library preparation can include normalization of a concentration of genetic material in each well of the sample microplate. Library preparation can further include fragmentation of the genetic material via an enzyme or via the application of physical forces. During this process, the entire genome (e.g., roughly three billion base pairs for a human genome) may be fragmented into pieces. In an example, the pieces can be about 300 to 400 base pairs in length. These pieces can be referred to as nucleic acid fragments. These nucleic acid fragments can undergo adaptor ligation and indexing. In an example, this can include Next Generation Sequencing (NGS) library preparation processes.

The genetic material can then be amplified, such as by Polymerase Chain Reaction (PCR) amplification. The resulting solution can be purified and eluted. During this library preparation, one or more reference samples of genetic material can be added to the wells of the library preparation microplate. The reference samples can serve as controls and aid in quality control.

Once the library preparation has been completed, thousands or millions of distinct fragments of the genetic material, each corresponding with a different portion of a genome of the subject, can be ligated to predefined adapters that bind with the genetic material. Each of the adaptor ligated fragments is referred to as a “library.”

In additional examples, probes applied to each well can include chemical identifiers (“barcodes”) that are distinct from each other. The use of a different chemical identifier for probes applied to each well of the well plate can enable sequencing to later be performed for multiple subjects on the same flow cell, without conflating sequencing results for those subjects.

In additional examples, the library preparation process can further include controlling a concentration of the genetic material in each well and purification, elution, or both, of the resulting material. Similar to the processes performed after extraction of genetic material, concentration of genetic material after library preparation can be confirmed for each well via testing.

Enrichment of Genetic Material. After library preparation, enrichment processes can be performed in order to either directly amplify (e.g., via amplicon or multiplexed PCR) or capture (e.g., via hybrid capture) predefined libraries of genetic material, such as at operation 926 in FIG. 11. This can enhance the ease of sequencing desired portions of the genome.

Here, mitochondrial probes can be used during genetic sample enrichment, prior to amplification, to capture mitochondrial DNA (mtDNA). Pathogen genetic material is also captured, via the use of separate, pathogen-specific probes, such as in a viral assay. The mtDNA is amplified and sequenced along with the pathogen genetic material. The sequenced mtDNA is collected and called to produce a plurality of reads. The plurality of reads are aligned to a mitochondrial contig (a set of overlapping DNA segments that together represent a consensus region of DNA).

For example, during enrichment, customized biotinylated oligonucleotide probes can be applied to the libraries. The probes can selectively hybridize genetic material occupying desired portions of the genome for the genetic material, such as specific genes, or the entire exome. Magnetic beads can bind to biotin molecules in the probes to attach the hybridized material to the magnetic beads. Magnetic forces can capture the beads in place, enabling remaining fluid within each well to be removed or washed out, thereby removing impurities, and leaving only the genetic material that is desired. Thus, genetic material can be released from the beads in a similar manner to that discussed above for prior processes.

In an example, hybrid capture target enrichment can be performed. During this process, the probes can include tailored oligonucleotides that are chosen to bind to the genetic material. The range of probes can be tailored as a group to bind to specific alleles, specific genes, the exome, the entire genome, or any suitable combination thereof. That is, each probe can bind to a nucleic acid fragment at a specific location on the genome, and the range of probes can be selected to ensure that alleles, genes, the exome, or the entire genome of the subject being considered is acquired.

In these examples, utilizing probes in this manner can enhance efficiency of the sequencing process, by foregoing the need to sequence all of the roughly three billion base pairs found in the human genome. The enrichment process can further include controlling a concentration of the genetic material in each well and purification, elution, or both, of the resulting material. Similar to the processes performed after extraction of genetic material, concentration of genetic material after enrichment can be confirmed for each well via testing.

Sequencing of Genetic Material. After enrichment, the genetic material can be sequenced at operation 928. Sequencing can be performed according to any of a variety of techniques, including short-read and long-read techniques.

In an example, the sequencing can be performed as Sequencing by Synthesis (SBS) at genetic analyzer equipment. For example, sets of enriched libraries of genetic material bound to probes in earlier operations can be transferred to a flow cell and annealed to oligonucleotide probes within the flow cell. At this stage, the contents of multiple wells can be applied to the same flow cell, because the libraries within those wells are tagged with the chemical identifiers referred to above.

In an example, the chemical identifiers can include nucleotide sequences that are detectable during the sequencing process to determine a corresponding LSI. Complementary sequences can then be created via enzymatic extension to create a double-stranded portion of genetic material. The double-stranded genetic material can then be denatured, and the library fragment can be washed away. Bridge amplification can then be performed to create copies of the remaining molecule in a localized cluster. For example, a cluster can comprise twenty to fifty copies of the same molecule, localized to a location that is smaller than a pinhead on the flow cell. Sequencing primers can be annealed to library adapters to prepare the flow cell for SBS. During SBS, the sequencing primer uses reverse terminator fluorescent oligonucleotides, one base per cycle, for several cycles in the forward direction. After the addition of each nucleotide, clusters can be excited by a light source, resulting in fluorescence that can be measured. The emission wavelength and signal intensity for each cluster determines a base call for that cluster. A chemical group blocking a 3′ end of the fragment can then be removed, enabling a subsequent nucleotide to be read. This can help control nucleotide addition and detection. After each cycle, denaturing and annealing can be performed to extend the index primer. A complementary reverse strand can be created and extended via bridge amplification. The reverse strand can then be read in the reverse direction for a number of cycles, in a manner similar to reads in the forward direction.

Different reagents can be chosen, depending on whether a complete human genome or another set of genomic data is being tested. That is, different reagents can be utilized for library preparation for a pathogen (e.g., bacteria, virus) or an organelle (e.g., mitochondria) than for a human genome. Pathogens exhibiting RNA genomes can have their genetic material translated to DNA before sequencing, enrichment, library preparation, or any suitable combination thereof, are performed.

In some examples, genetic material can be used for detection of a pathogen rather than for sequencing. Detecting a pathogen can involve the use of a real-time PCR system that performs PCR. The real-time PCR system can further add a reactive agent to individual wells of a library preparation microplate that fluoresces when bound to genetic material for the pathogen. By analyzing fluorescence at known periods of time after PCR has initiated, presence of a pathogen is determined. Genetic testing for a pathogen can thereby forego sequencing in some examples.

Throughout the processes discussed above, the laboratory environment can be carefully controlled to ensure quality. For example, temperature within each segment of the laboratory can be carefully monitored and controlled, and ultraviolet lighting or other features capable of inactivating genetic material can be carefully positioned to ensure that contamination does not occur.

In general, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text based FASTQ format, that reports quality scores. The second format is then analyzed to perform alignment of sequence reads to a reference genome, such as a reference genome reported in a Browser Extensible Data (BED) file. The aligned sequence data may be reported as a BAM file. The aligned sequence data may then be analyzed further (e.g., called) using a variant calling process, resulting in a VCF file reporting called variants at each location of the genome that was sequenced, together with secondary metrics such as quality indicator metrics. The called sequence data may be provided to a data analyst via a user interface (UI), such as a graphical user interface (GUI) presented via a display. The technician may then validate the resulting called sequence data (e.g., with or without associated metrics) and release it for reporting to subjects, health care providers, scientists, or any suitable combination thereof.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and their functionality presented as separate components and functions in example configurations may be implemented as a combined structure or component with combined functions. Similarly, structures and functionality presented as a single component may be implemented as separate components and functions. These and other variations, modifications, additions, and improvements fall within the scope of the subject matter herein.

Some portions of the subject matter discussed herein may be presented in terms of algorithms or symbolic representations of operations on data stored as bits or binary digital signals within a memory (e.g., a computer memory or other machine memory). Such algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “accessing,” “processing,” “detecting,” “computing,” “calculating,” “determining,” “generating,” “presenting,” “displaying,” or the like refer to actions or processes performable by a machine (e.g., a computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non-exclusive “or,” unless specifically stated otherwise.

The following enumerated descriptions describe various examples of methods, machine-readable media, and systems (e.g., machines, devices, or other apparatus) discussed herein. Any one or more features of an example, taken in isolation or combination, should be considered as being within the disclosure of this application.

A first example provides a method comprising: accessing, by one or more processors of a machine, first results of a first analysis performed by a first version of a first analytical tool on sequenced data of a genetic sample, a second analytical tool being available to process results of the first analytical tool;
determining, by the one or more processors of the machine, that a second analysis is to be performed by a second version of the first analytical tool upon the sequenced data of the genetic sample, a third analytical tool into which second results of the second analytical tool are to be inputted specifying a minimum version of the first analytical tool, the determining that the second analysis is to be performed being based on the first version failing to satisfy the minimum version in conjunction with the second version satisfying the minimum version; and
causing, by one or more processors of the machine, the second analysis to be performed by the second version of the first analytical tool on the sequenced data of the genetic sample.

A second example provides a method according to the first example, wherein:
the second results of the second analysis by the first analytical tool include an improvement absent from the first results of the first analysis by the first analytical tool, the improvement including at least one of a correction to an error, an additional datum, a datum with increased precision, or a datum with increased accuracy.

A third example provides a method according to the first example or the second example, wherein:
the second analysis by the first analytical tool includes an improvement absent from the first analysis by the first analytical tool, the improvement including at least one of analyzing an additional portion of a gene, analyzing an additional gene, omitting analysis of a portion of a gene, omitting analysis of a gene, analyzing more total portions of genes, analyzing more total genes, analyzing fewer total portions of genes, or analyzing fewer total genes.

A fourth example provides a method according to any of the first through third examples, wherein:
the second results of the second analysis by the first analytical tool include an improvement absent from the first results of the first analysis by the first analytical tool, the improvement including at least one of a data format supported by the third analytical tool, a file format supported by the third analytical tool, a metadata type supported by the third analytical tool, or a metadata value supported by the third analytical tool.

A fifth example provides a method according to any of the first thorough fourth examples, wherein:
the first analytical tool is a primary analysis tool configured to perform alignment of the sequenced data of the genetic sample.

A sixth example provides a method according to any of the first through fifth examples, wherein:
the second analytical tool is a secondary analysis tool configured to perform variant calling on the first results of the first analytical tool.

A seventh example provides a method according to any of the first through sixth examples, wherein:
the third analytical tool is a tertiary analysis tool configured to process secondary results outputted by the second analytical tool based on processing the first results of the first analytical tool.

An eighth example provides a method according to the seventh example, wherein:
the tertiary analysis tool is configured to process the secondary results by performing at least one of ancestry analysis, traits analysis, or medical diagnosis.

A ninth example provides a machine-readable medium (e.g., a non-transitory machine-readable storage medium) comprising instructions that, when executed by one or more processors of a machine, cause the machine to perform operations comprising:
accessing first results of a first analysis performed by a first version of a first analytical tool on sequenced data of a genetic sample, a second analytical tool being available to process results of the first analytical tool;
determining that a second analysis is to be performed by a second version of the first analytical tool upon the sequenced data of the genetic sample, a third analytical tool into which second results of the second analytical tool are to be inputted specifying a minimum version of the first analytical tool, the determining that the second analysis is to be performed being based on the first version failing to satisfy the minimum version in conjunction with the second version satisfying the minimum version; and
causing the second analysis to be performed by the second version of the first analytical tool on the sequenced data of the genetic sample.

A tenth example provides a machine-readable medium according to the ninth example, wherein:
the second results of the second analysis by the first analytical tool include an improvement absent from the first results of the first analysis by the first analytical tool, the improvement including at least one of a correction to an error, an additional datum, a datum with increased precision, or a datum with increased accuracy.

An eleventh example provides a machine-readable medium according to the ninth example or the tenth example, wherein:
the second analysis by the first analytical tool includes an improvement absent from the first analysis by the first analytical tool, the improvement including at least one of analyzing an additional portion of a gene, analyzing an additional gene, omitting analysis of a portion of a gene, omitting analysis of a gene, analyzing more total portions of genes, analyzing more total genes, analyzing fewer total portions of genes, or analyzing fewer total genes.

A twelfth example provides a machine-readable medium according to any of the ninth through eleventh examples, wherein:
the second results of the second analysis by the first analytical tool include an improvement absent from the first results of the first analysis by the first analytical tool, the improvement including at least one of a data format supported by the third analytical tool, a file format supported by the third analytical tool, a metadata type supported by the third analytical tool, or a metadata value supported by the third analytical tool.

A thirteenth example provides a machine-readable medium according to any of the ninth through twelfth examples, wherein:
the first analytical tool is a primary analysis tool configured to perform alignment of the sequenced data of the genetic sample.

A fourteenth example provides a machine-readable medium according to any of the ninth through thirteenth examples, wherein:
the second analytical tool is a secondary analysis tool configured to perform variant calling on the first results of the first analytical tool.

A fifteenth example provides a machine-readable medium according to any of the ninth through fourteenth examples, wherein:
the third analytical tool is a tertiary analysis tool configured to process secondary results outputted by the second analytical tool based on processing the first results of the first analytical tool.

A sixteenth example provides a system (e.g., a computer system, one or more computers, a machine, other apparatus, or any suitable combination thereof) comprising:
one or more processors; and
a memory storing instructions that, when executed by at least one processor among the one or more processors, cause the system to perform operations comprising:
accessing first results of a first analysis performed by a first version of a first analytical tool on sequenced data of a genetic sample, a second analytical tool being available to process results of the first analytical tool;
determining that a second analysis is to be performed by a second version of the first analytical tool upon the sequenced data of the genetic sample, a third analytical tool into which second results of the second analytical tool are to be inputted specifying a minimum version of the first analytical tool, the determining that the second analysis is to be performed being based on the first version failing to satisfy the minimum version in conjunction with the second version satisfying the minimum version; and
causing the second analysis to be performed by the second version of the first analytical tool on the sequenced data of the genetic sample.

A seventeenth example provides a system according to the sixteenth example, wherein:
the second results of the second analysis by the first analytical tool include an improvement absent from the first results of the first analysis by the first analytical tool, the improvement including at least one of a correction to an error, an additional datum, a datum with increased precision, or a datum with increased accuracy.

An eighteenth example provides a system according to the sixteenth example or the seventeenth example, wherein:
the second analysis by the first analytical tool includes an improvement absent from the first analysis by the first analytical tool, the improvement including at least one of analyzing an additional portion of a gene, analyzing an additional gene, omitting analysis of a portion of a gene, omitting analysis of a gene, analyzing more total portions of genes, analyzing more total genes, analyzing fewer total portions of genes, or analyzing fewer total genes.

A nineteenth example provides a system according to any of the sixteenth through eighteenth examples, wherein:
the second results of the second analysis by the first analytical tool include an improvement absent from the first results of the first analysis by the first analytical tool, the improvement including at least one of a data format supported by the third analytical tool, a file format supported by the third analytical tool, a metadata type supported by the third analytical tool, or a metadata value supported by the third analytical tool.

A twentieth example provides a system according to any of the sixteenth through nineteenth examples, wherein:
the first analytical tool is a primary analysis tool configured to perform alignment of the sequenced data of the genetic sample;
the second analytical tool is a secondary analysis tool configured to perform variant calling on the first results of the first analytical tool; and
the third analytical tool is a tertiary analysis tool configured to process secondary results outputted by the second analytical tool based on processing the first results of the first analytical tool.

A twenty-first example provides a carrier medium carrying machine-readable instructions for controlling a machine to carry out the operations (e.g., method operations) performed in any one of the previously described examples.

What is claimed is:

1. A method comprising:
accessing, by one or more processors of a machine, first results of a first analysis performed by a first version of a first analytical tool on sequenced data of a genetic sample, a second analytical tool being available to process results of the first analytical tool;
determining, by the one or more processors of the machine, that a second analysis is to be performed by a second version of the first analytical tool upon the sequenced data of the genetic sample, a third analytical tool into which second results of the second analytical tool are to be inputted specifying a minimum version of the first analytical tool, the determining that the second analysis is to be performed being based on the first version failing to satisfy the minimum version in conjunction with the second version satisfying the minimum version; and
causing, by one or more processors of the machine, the second analysis to be performed by the second version of the first analytical tool on the sequenced data of the genetic sample.

2. The method of claim 1, wherein:
the second results of the second analysis by the first analytical tool include an improvement absent from the first results of the first analysis by the first analytical tool, the improvement including at least one of a correction to an error, an additional datum, a datum with increased precision, or a datum with increased accuracy.

3. The method of claim 1, wherein:
the second analysis by the first analytical tool includes an improvement absent from the first analysis by the first analytical tool, the improvement including at least one of analyzing an additional portion of a gene, analyzing an additional gene, omitting analysis of a portion of a gene, omitting analysis of a gene, analyzing more total portions of genes, analyzing more total genes, analyzing fewer total portions of genes, or analyzing fewer total genes.

4. The method of claim 1, wherein:
the second results of the second analysis by the first analytical tool include an improvement absent from the first results of the first analysis by the first analytical tool, the improvement including at least one of a data format supported by the third analytical tool, a file format supported by the third analytical tool, a metadata type supported by the third analytical tool, or a metadata value supported by the third analytical tool.

5. The method of claim 1, wherein:
the first analytical tool is a primary analysis tool configured to perform alignment of the sequenced data of the genetic sample.

6. The method of claim 1, wherein:
the second analytical tool is a secondary analysis tool configured to perform variant calling on the first results of the first analytical tool.

7. The method of claim 1, wherein:
the third analytical tool is a tertiary analysis tool configured to process secondary results outputted by the second analytical tool based on processing the first results of the first analytical tool.

8. The method of claim 7, wherein:
the tertiary analysis tool is configured to process the secondary results by performing at least one of ancestry analysis, traits analysis, or medical diagnosis.

9. A non-transitory machine-readable storage medium comprising instructions that, when executed by one or more processors of a machine, cause the machine to perform operations comprising:
accessing first results of a first analysis performed by a first version of a first analytical tool on sequenced data of a genetic sample, a second analytical tool being available to process results of the first analytical tool;
determining that a second analysis is to be performed by a second version of the first analytical tool upon the sequenced data of the genetic sample, a third analytical tool into which second results of the second analytical tool are to be inputted specifying a minimum version of the first analytical tool, the determining that the second analysis is to be performed being based on the first version failing to satisfy the minimum version in conjunction with the second version satisfying the minimum version; and
causing the second analysis to be performed by the second version of the first analytical tool on the sequenced data of the genetic sample.

10. The non-transitory machine-readable storage medium of claim 9, wherein:
the second results of the second analysis by the first analytical tool include an improvement absent from the first results of the first analysis by the first analytical tool, the improvement including at least one of a correction to an error, an additional datum, a datum with increased precision, or a datum with increased accuracy.

11. The non-transitory machine-readable storage medium of claim 9, wherein:
the second analysis by the first analytical tool includes an improvement absent from the first analysis by the first analytical tool, the improvement including at least one of analyzing an additional portion of a gene, analyzing an additional gene, omitting analysis of a portion of a gene, omitting analysis of a gene, analyzing more total portions of genes, analyzing more total genes, analyzing fewer total portions of genes, or analyzing fewer total genes.

12. The non-transitory machine-readable storage medium of claim 9, wherein:
the second results of the second analysis by the first analytical tool include an improvement absent from the first results of the first analysis by the first analytical tool, the improvement including at least one of a data format supported by the third analytical tool, a file format supported by the third analytical tool, a metadata type supported by the third analytical tool, or a metadata value supported by the third analytical tool.

13. The non-transitory machine-readable storage medium of claim 9, wherein:
the first analytical tool is a primary analysis tool configured to perform alignment of the sequenced data of the genetic sample.

14. The non-transitory machine-readable storage medium of claim 9, wherein:
the second analytical tool is a secondary analysis tool configured to perform variant calling on the first results of the first analytical tool.

15. The non-transitory machine-readable storage medium of claim 9, wherein:
the third analytical tool is a tertiary analysis tool configured to process secondary results outputted by the second analytical tool based on processing the first results of the first analytical tool.

16. A system comprising:
one or more processors; and
a memory storing instructions that, when executed by at least one processor among the one or more processors, cause the system to perform operations comprising:
accessing first results of a first analysis performed by a first version of a first analytical tool on sequenced data of a genetic sample, a second analytical tool being available to process results of the first analytical tool;
determining that a second analysis is to be performed by a second version of the first analytical tool upon the sequenced data of the genetic sample, a third analytical tool into which second results of the second analytical tool are to be inputted specifying a minimum version of the first analytical tool, the determining that the second analysis is to be performed being based on the first version failing to satisfy the minimum version in conjunction with the second version satisfying the minimum version; and
causing the second analysis to be performed by the second version of the first analytical tool on the sequenced data of the genetic sample.

17. The system of claim 16, wherein:
the second results of the second analysis by the first analytical tool include an improvement absent from the first results of the first analysis by the first analytical tool, the improvement including at least one of a correction to an error, an additional datum, a datum with increased precision, or a datum with increased accuracy.

18. The system of claim 16, wherein:
the second analysis by the first analytical tool includes an improvement absent from the first analysis by the first analytical tool, the improvement including at least one of analyzing an additional portion of a gene, analyzing an additional gene, omitting analysis of a portion of a gene, omitting analysis of a gene, analyzing more total portions of genes, analyzing more total genes, analyzing fewer total portions of genes, or analyzing fewer total genes.

19. The system of claim 16, wherein:
the second results of the second analysis by the first analytical tool include an improvement absent from the first results of the first analysis by the first analytical tool, the improvement including at least one of a data format supported by the third analytical tool, a file format supported by the third analytical tool, a metadata type supported by the third analytical tool, or a metadata value supported by the third analytical tool.

20. The system of claim 16, wherein:
the first analytical tool is a primary analysis tool configured to perform alignment of the sequenced data of the genetic sample;
the second analytical tool is a secondary analysis tool configured to perform variant calling on the first results of the first analytical tool; and
the third analytical tool is a tertiary analysis tool configured to process secondary results outputted by the second analytical tool based on processing the first results of the first analytical tool.
